(19) United States
(12) Patent Application Publication
(10) Pub. No.: US 2002/0114834 A1
(43) Pub. Date: Aug. 22, 2002

(54) Method of dispensing a formula with medical or nutritional preparations in the form of initially-reduced-release particles (IRRP)

(76) Inventor: Lih-Min Her, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/733,979

(22) Filed: Dec. 12, 2000

(51) Int. Cl. A61K 9/22; A61K 35/78

(52) U.S. Cl. 424/468; 424/725

(57) ABSTRACT

A method of dispensing a formula with medical or nutritional preparations in the form of initially-reduced-release particles (IRRP) is provided. In the method, one or more types of IRRP medicines or galenicals or herbal supplements are used in dispensing a formula or preparing a new medicine in which contents of one or more types of medicines or galenicals or herbal supplements meet a patient's or a user's individual need. The IRRP have diameters within the range from 0.05 mm to 4.00 mm and are covered with one or more layers of coatings or thin films or surroundings to prevent, reduce or delay release of the medicines or galenicals or herbal supplements contained in the IRRP in water of pH 7.0±0.1 at 37° C.

### BACKGROUND OF THE INVENTION

The present invention relates to a method of dispensing a formula with medical or nutritional preparations in the form of initially-reduced-release particles (IRRP), and more particularly to a method of dispensing a formula with coated IRRP of medicines, galenicals or herbal supplements to facilitate the dispensing and increase the medicine stability.

Currently, it is a common practice in most large-scaled hospitals to dispense a formula of scientific Chinese medicines by using powdered Chinese medicine extracts or pulverized Chinese medicines, mixing one or more types of the pulverized Chinese medicines and dividing the resultant mixture into predetermined number of doses for a patient to take in a specified manner and at specified time intervals. The above-described conventional method of dispensing the formula of Chinese medicines has the following disadvantages:

1. The scientific Chinese medicines usually taste bad and include a large number of doses that discourages patients from consistently taking medicines.

2. Powdered scientific Chinese medicines directly contact with air in large contact area and are therefore subject to deliquescence, oxidization or deterioration and have shortened shelf life.

3. The pulverized scientific Chinese medicines contain large amount of fine powders that tends to degrade the quality of medical or nutritional preparations and pollute a pharmacist's work environment, as well as cause cross-contamination among different medicines during dispensing the formulae.

4. Some of the scientific Chinese medicines are in the form of capsule and tablet having fixed ingredients and dosage and are therefore not suitable for accurate dispensing to meet a patient's individual need. Moreover, it is difficult to swallow capsuled and tablet medicines for some patients.

As to general medicines, galenicals or herbal supplements frequently used in western countries, they also have the following drawbacks:

1. The general medicines are usually in the form of capsule or tablet having fixed dosage that is not suitable for accurate preparation of medicines to satisfy a patient's individual need.

2. Some patients have difficulty in swallowing capsules and tablets. In the event the capsules are opened or the tablets are ground to powder form for easy taking by patients, the medicines usually taste bad to discourage the patients.

3. When more than on type of medicines, galenicals or herbal supplements are to be taken and each type of them has different number of tablets and capsules contained in one dose, the patient tends to be confused with the medicines.

### SUMMARY OF THE INVENTION

It is therefore a primary object of the present invention to provide a method of dispensing a formula with medical or nutritional preparations in the form of initially-reduced-release particles (IRRP), so that a formula containing one or more types of medicines, galenicals or herbal supplements can be dispensed to meet each patient's or user's need. Medical or nutritional preparations in the form of IRRP also have increased medicine stability and shelf life.

To achieve the above and other objects, the initially-reduced-release particles (IRRP) of medical or nutritional preparations used in the present invention have diameters within the range from 0.05 mm to 4 mm and are covered with one or more layers of coatings or thin films or surroundings. The coating or thin film or surroundings used in the present invention is mainly made of, but not limited to, non-water-soluble, low-water-soluble, acid-soluble, alkali-soluble, hydrogel forming, slowly-dissolving, biodegradable, hydrophilic, lipophilic, water-soluble, fat-soluble, and/or flavor-improving substance, and/or any other equivalent substance.

The coating or thin film or surroundings used in the present invention may be including, but not limited to, semi-permeable, permeable, gastric, enteric, erodible, slowly dissolving, biodegradable, low-water-soluble and/or partially dissolving coating and/or any other coating having similar nature.

With respect to the above-mentioned IRRP of medicine, galenicals or herbal supplements, less than 10% of the content of the medicine, galenicals or herbal supplements would release in water of pH 7.0±0.1 at 37° C. within the first one minute.

In the present invention, the above-mentioned medicines, galenicals or herbal supplements are made into particles and are covered with coatings or thin films or surroundings.

### BRIEF DESCRIPTION OF THE DRAWINGS

The above objects and advantages of the present invention will become more apparent by describing in details some preferred embodiments thereof with reference to the accompanying drawings, wherein

FIG. 1 is a flowchart showing steps included in the method according to a first embodiment of the present invention for dispensing a formula with medical or nutritional preparations in the form of initially-reduced-release particles (IRRP);

FIG. 2 is a flowchart showing steps included in the method according to a second embodiment of the present invention for dispensing a formula with medical or nutritional preparations in the form of IRRP; and

FIG. 3 is a flowchart showing steps included in the method according to a third embodiment of the present invention for dispensing a formula with medical or nutritional preparations in the form of IRRP.

### DETAILED DESCRIPTION OF THE PREFERRED EMBODIMENTS

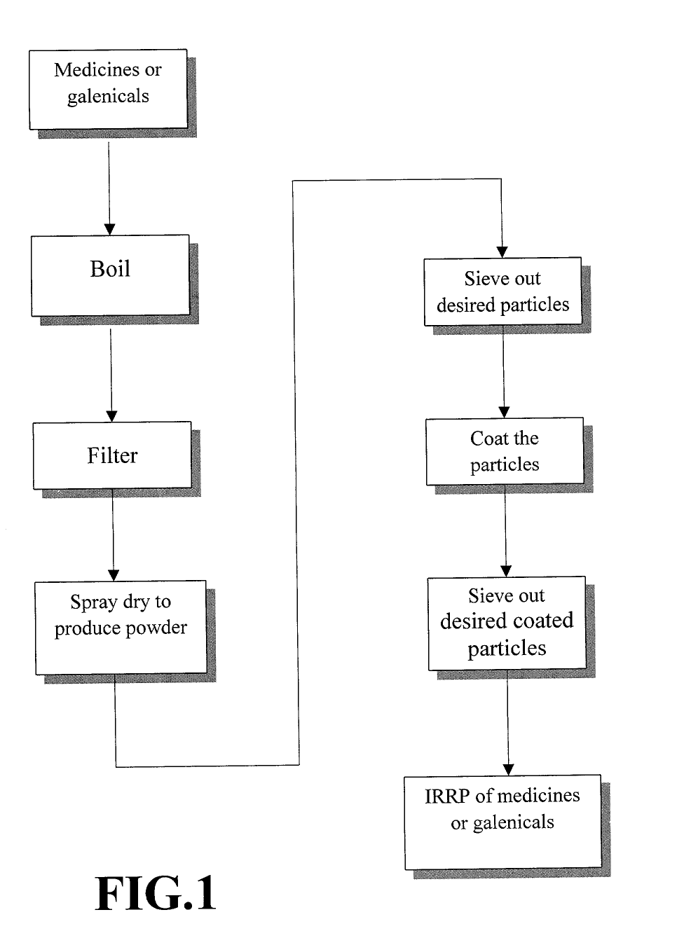

Please refer to FIG. 1 in which steps for producing medical or nutritional preparations in the form of initially-reduced-release particles (IRRP) according to a first embodiment of the present invention are shown. These steps can be understood by referring to the detailed descriptions of following examples of producing IRRP of different medical or nutritional preparations.

### EXAMPLE 1

### IRRP of Grape-Seed Extract

1. Add 12,000 g of grape seeds into 54,000 g of alcohol-water blend containing 5% alcohol and boil the mixture for three hours. The boiled mixture is filtered and the resultant filtrate is spray dried to produce dried powder of grape-seed extract.

2. Use No. 100 sieve (0.149 mm) and No. 270 sieve (0.053 mm) to sieve the dried powder of grape-seed extract obtained in the step 1, so that particles of grape-seed extract sized between 0.053 mm and 0.149 mm are obtained. Use a Fluidized-Bed Coater Model Glatt® GPCG 1 to coat 160 g of sieved dry particles with 1600 ml of solution containing 2% Cellulose acetate, 0.5% Eudrogit® E100 and 97.5% acetone.

3. Sieve the coated particles with No. 80 sieve (0.177 mm) and No. 270 sieve (0.053 mm) to obtain particles sized between 0.053 mm and 0.177 mm that form the desired IRRP of grape-seed extract. The IRRP of grape-seed extract so produced contains 80% grape-seed extract by weight.

The IRRP of grape-seed extract produced in the above example 1 has a coating or thin film or surrounding that mainly contains non-water-soluble, low-water-soluble, acid-soluble, alkali-soluble, gelled-with-water, slowly-dissolving, biodegradable, hydrophilic, lipophilic, water-soluble, fat-soluble, and/or flavor-improving substances and/or any other equivalent substance. And, such coating or thin film or surrounding is semi-permeable, permeable, gastric, enteric, erodible, slowly dissolving, biodegradable, low-water-soluble and/or partially dissolving and/or has any other similar nature. And, the IRRP of medicines, galenicals or herbal supplements have diameters between 0.05 mm and 4 mm. And, less than 10% of the medicines, galenicals or herbal supplements contained in the IRRP would release in water of pH 7.0±0.1 at 37° C. in the first one minute.

### EXAMPLE 2

### IRRP of Korean Ginseng Extract

1. Add 60,000 g of Korean ginseng into 180,000 g of water and boil the mixture for three hours. The boiled mixture is filtered and the resultant filtrate is concentrated, so that a Korean ginseng concentrate containing 10% Korean ginseng extract is obtained.

2. Add 20 g of polyvinyl pyrrolidone (PVP) into 2,000 cc of Korean ginseng concentrate, and heat while stirring the mixture until the PVP therein is fully dissolved.

3. Sieve sugar granules with No. 30 sieve (0.59 mm) and No. 40 sieve (0.42 mm) to select granules sized between 0.42 mm and 0.59 mm, Weigh out 780 g of sieved sugar granules. Use a Fluidized-Bed Coater model Glatt® GPCG 1 to spray the PVP/Korean ginseng concentrate prep are d in the step 2 over the sieved sugar granules to coat the latter to obtain desired Korean ginseng extract granules.

4. Coat 150 g of Korean ginseng extract granules with 1000 ml of solution containing 5% Eudragit® S100 and 95% isopropanol by spraying the solution over the granules.

5. Dry the coated granules and sieve them with No. 18 sieve (1.00 mm) and No. 40 sieve (0.42 mm) to sieve out desired granules sized between 0.42 mm and 1.00 mm. The sieved granules are the desired IRRP of Korean ginseng extract containing about 15% Korean ginseng extract by weight.

### EXAMPLE 3

### IRRP of Giant Knot Rhizome and grape seeds

1. Add 12,000 g of Giant Knot Rhizome into 54,000 g of alcohol-water blend containing 5% alcohol and boil the mixture for three hours. The boiled mixture is filtered and the resultant filtrate is evaporated and thereby concentrated to produce a concentrate containing 10% Giant Knot Rhizome extract. Add 12,000 g of grape seeds into 54,000 g of alcohol-water blend containing 5% alcohol and boil the mixture for three hours. The boiled mixture is filtered and the resultant filtrate is evaporated and thereby concentrated to produce a concentrate containing 10% grape-seed extract. Mix 1,000 g of Giant Knot Rhizome concentrate with 500 g of grape-seed concentrate. The mixture is filtered and the resultant filtrate is added with 350 g of cornstarch. The new mixture is spray dried to produce powder. Sieve the powder with No. 40 sieve (0.42 mm).

2. Sieve the powder with No. 40 sieve (0.42 mm) and No. 120 sieve (0.125 mm) to sieve out particles sized between 0.125 mm and 0.42 mm. Use a Fluidized-Bed Coater model Glatt® GPCG 1 to spray 2000 ml of solution containing 2.25% ethyl cellulose, 0.25% hydroxypropylmethylcellulose, and 97.5% isopropanol over 200 g of the sieved dry particles to coat the same.

3. Dry the coated particles and sieve them with No. 30 sieve (0.59 mm) and No. 120 sieve (0.125 mm) to sieve out particles sized between 0.125 mm and 0.59 mm that are the desired IRRP of Giant Knot Rhizome and grape seeds. The IRRP of Giant Knot Rhizome and grape seeds so produced contain about 16% Giant Knot Rhizome extract and about 8% grape-seed extract by weight.

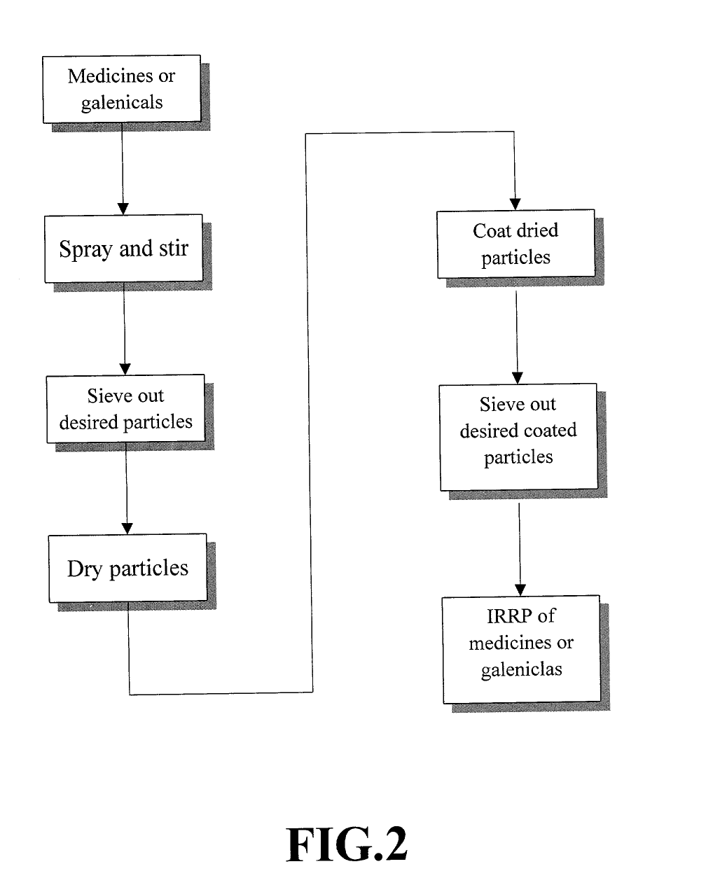

FIG. 2 shows steps for producing IRRP of medical or nutritional preparations in the method according to a second embodiment of the present invention. These steps can be understood by referring to the detailed descriptions of following examples of producing IRRP of different medical or nutritional preparations.

### EXAMPLE 4

### IRRP of Aspirin

1. Use 200 ml of PVP-water solution containing 10% PVP to spray over 480 g of Aspirin and stir the wetted Aspirin. Sieve the wetted and stirred Aspirin with No. 45 sieve (0.35 mm) and dry the sieved Aspirin in a drying oven at 60° C. for 48 hours.

2. Sieve the dried Aspirin again with No. 45 sieve (0.35 mm) and No. 120 sieve (0.125 mm) to sieve out particles of Aspirin sized between 0.125 mm and 0.35 mm. Use a Fluidized-Bed Coater model Glatt® GPCG 1 to coat 150 g of sieved dry particles of Aspirin with 1000 ml of solution containing 5% Eudragit® S100 and 95% isopropanol.

3. Sieve the coated particles with No. 35 sieve (0.50 mm) and No. 120 sieve (0.125 mm) to sieve out particles sized between 0.125 mm and 0.50 mm. The sieved particles are the desired IRRP of Aspirin containing about 72% Aspirin by weight.

### EXAMPLE 5

### IRRP of Four-Ingredients (Including Prepared Chinese Foxglove Root, Chinese Angelica Root, White Peony Root, and Chuanxiong Rhizome)

1. Mix 960 g of prepared Chinese foxglove root, 960 g of Chinese Angelica root, 640 g of white peony root, 480 g of Chuanxiong Rhizome, and 760 g of micro-crystalline cellulose. Add about 3,000 g of water into the mixture and fully stir the same. The fully stirred mixture is extruded through an extrusion spheronizer to form pellets that are then dried in a drying oven at 60° C. for 48 hours.

2. Sieve the pellets with No. 10 sieve (2.00 mm) and No. 20 sieve (0.84 mm) to sieve out pellets sized between 0.84 mm and 2.00 mm. Use a Fluidized-Bed Coater model Glatt® GPCG 1 to coat 200 g of sieved dry pellets with 1000 ml of solution containing 5% Eudragit® E100 and 95% isopropanol by spraying the solution over the pellets.

3. Sieve the coated pellets with No. 6 sieve (3.36 mm) and No. 20 sieve (0.84 mm) to sieve out pellets sized between 0.84 mm and 3.36 mm for forming the IRRP of Four-Ingredients. The IRRP of Four-Ingredients so produced contains 64% prepared Chinese foxglove root, Chinese Angelica root, white peony root and Chuanxiong Rhizome by weight.

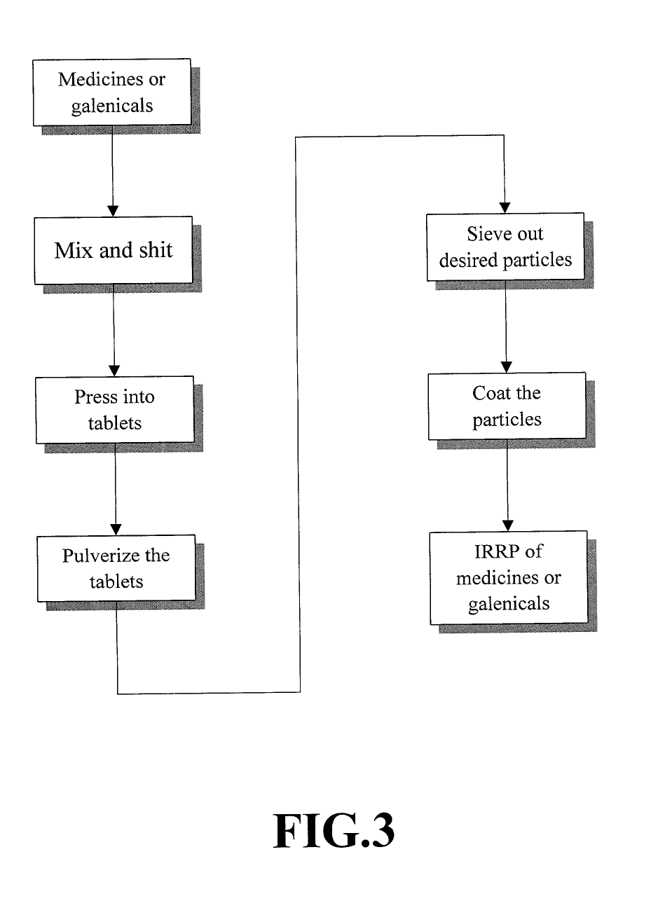

FIG. 3 shows steps for producing IRRP of medical or nutritional preparations in the method according to a third embodiment of the present invention. These steps can be understood by referring to the detailed descriptions of following examples of producing IRRP of different medical or nutritional preparations.

### EXAMPLE 6

### IRRP of Chloropheniramine

1. Use a No. 200 sieve to separately sieve 10 g of chloropheniramine powder and 990 g of calcium carbonate powder. Mix the two types of sieved powder and press the mixture with a tablet press into tablets of 2 cm in diameter. Breaking these tablets into particles and sieve the particles.

2. Sieve the particles with No. 16 sieve (1.19 mm) and No. 30 sieve (0.59 mm) to sieve out particles having sizes between 0.59 mm and 1.19 mm that form the desired IRRP of chloropheniramine.

3. Coat 200 g of the IRRP of chloropheniramine with 1000 g of water solution containing 4% maltose and 1% green tea powder. Dry the coated particles and sieve them with No. 10 sieve (2.00 mm) and No. 30 sieve (0.59 mm) to sieve out particles sized between 0.59 mm and 2.00 mm that are the desired green-tea-flavored IRRP of chloropheniramine containing about 0.8% chloropheniramine by weight.

In measuring the release amount of the above-described various types of IRRP of medical or nutritional preparations, use a paddle to stir 500 ml of water of 37° C. and pH 7.0±0.1 in a circular movement at a speed of 50 rpm, and then put 1 g of tested IRRP of medical preparation into the water. Wait for one minute and sample 10 ml of the resultant water. Test and measure contents of medicines and/or galenicals and/or herbal supplements in the sampled water, and calculate the ratio of released medicines, galenicals or herbal supplements to the tested IRRP medical preparation. The following are release ratios found in the above-described examples of IRRP of different medical or nutritional preparations:

1. IRRP of grape-seed extract: 8.3%.

2. IRRP of Korean ginseng extract: 0%.

3. IRRP of Giant Knot Rhizome and grape seeds: 3.5% for grape-seed extract and 3.2% for Giant Knot Rhizome extract.

4. IRRP of Aspirin: 2.3%.

5. IRRP of Four-Ingredients: 0%.

6. IRRP of chloropheniramine: 0%.

The contents of major ingredients in the IRRP of medical or nutritional preparations can be adjusted by adjusting a total amount of the IRRP before using the same to dispense a formula and prepare doses for individual patients. For example, fully mix 100 mg of IRRP of Korean ginseng extract that contains about 15% Korean ginseng extract by weight with 1400 mg of cornstarch particles sized between No. 18 sieve (1.00 mm) and No. 40 sieve (0.42 mm). Particles of the resultant mixture shall contain only 1% Korean ginseng extract by weight. That is, the content of Korean ginseng extract in the original IRRP of Korean ginseng is diluted by 15 times.

The IRRP of medical or nutritional preparations produced in the above-described steps may be used in dispensing different formulae. The following are some examples of these formulae:

Formula 1:

Grape-seed extract 2 mg/kg; Korean ginseng extract 0.2 mg/kg; Giant Knotweed Rhizome extract 4 mg/kg; vitamin C 5 mg/kg; powdered licorice 20 mg/kg; Zinc glucuronate 0.5 mg/kg; and cornstarch particles q.s. to 5 g per dose; t.i.d. for 7 days.

For a subject A having a weight of 54 kg, the above formula may be dispensed as below:

| Medicine/Galenical | Content | Required | Total |
|---|---|---|---|
| prescribed | (% wt) | amount | weight |
| IRRP of grape-seed | 10 | 2.268 g | 22.68 g |
| extract | | | |
| IRRP of Korean | 1 | 0.2268 g | 22.68 g |
| ginseng extract | | | |
| IRRP of Giant | 20 | 4.536 g | 22.68 g |
| knotweed Rhizome | | | |
| extract | | | |
| Vitamin C | 100 | 5.67 g | 5.67 g |
| Powdered licorice | 100 | 22.68 g | 22.68 g |
| Zinc glucuronate | 10 | 0.567 g | 5.67 g |
| Cornstarch | 100 | 2.94 g | 2.94 g |
| Total weight | | | 105 g |

Prepare the above prescribed medicines and/or galenicals and/or herbal supplements and/or excipients, if needed, as per the weights shown in the last column of the above table and mix them to obtain 105 g of mixture of all prescribed medicines and/or galenicals and/or herbal supplements and/or excipients, if needed. The mixture is then divided by weight into 21 doses of 5 g each.

Formula 2:

Grape-seed extract 2 mg/kg; and glucose q.s. to 3 g per dose; t.i.d. for 7 days.

For a subject B having a weight of 63 kg, the above formula may be dispensed as below:

| Medicine/Galenical | Content | Required | Total |
|---|---|---|---|
| Prescribed | (% wt) | amount | weight |
| IRRP of grape-seed | 10 | 2.646 g | 26.46 g |
| extract | | | |
| Cornstarch particles | 100 | 36.54 g | 36.54 g |
| Total weight | | | 63 g |

Prepare the above prescribed medicines and/or galenicals and/or herbal supplements and/or excipients, if needed, as per the weights shown in the last column of the above table and mix them to obtain 63 g of mixture of all prescribed medicines and/or galenicals and/or herbal supplements and/or excipients, if needed. The mixture is then transferred into a measuring cylinder to measure the volume thereof. The measured mixture is then divided by volume into 21 doses of about 3 g each.

Formula 3:

Aspirin 3.5 mg/kg; Chlorophoniramic 0.07 mg/kg; and glucose particles q.s. to 3 g per dose; t.i.d. for 3 days.

For a subject C having a weight of 36 kg, the above formula may be dispensed as below:

| Medicine/Galenical | Content | Required | Total |
|---|---|---|---|
| Prescribed | (% wt) | amount | weight |
| IRRP of Aspirin | 10 | 1.134 g | 11.34 g |
| IRRP of | 0.4 | 0.02268 g | 5.67 g |
| Chloropheniramine | | | |
| Sucrose particles | 100 | 9.99 g | 9.99 g |
| Total weight | | | 27 g |

Prepare the above prescribed medicines and/or galenicals and/or herbal supplements and/or excipients, if needed, as per the weights shown in the last column of the above table and mix them to obtain 27 g of mixture of all prescribed medicines and/or galenicals and/or herbal supplements and/or excipients, if needed. The mixture is then divided by weight into 9 doses of 3 g each.

Formula 4:

Four-Ingredients (including prepared Chinese foxglove root, Chinese Angelica root, white peony root, and Chuanxiong Rhizome) 64 mg/kg; Korean ginseng extract 0.2 mg/kg; and licorice particles q.s. t 6 g per dose; t.i.d. for 7 days.

For a subject D having a weight of 47.5 kg, the above formula may be dispensed as below:

| Medicine/Galenical | Content | Required | Total |
|---|---|---|---|
| Prescribed | (% wt) | amount | weight |
| IRRP of | 64% | 63.84 g | 99.75 g |
| Four-Ingredients | | | |
| IRRP of Korean | 1% | 0.1995 g | 19.95 g |
| ginseng extract | | | |
| Licorice particles | 100% | 6.30 g | 6.30 g |
| Total weight | | | 126 g |

Prepare the above prescribed medicines and/or galenicals and/or herbal supplements and/or excipients, if needed, as per the weights shown in the last column of the above table and mix them to obtain 126 g of mixture of all prescribed medicines and/or galenicals and/or herbal supplements and/or excipients, if needed. The mixture is then divided by weight into 21 doses of 6 g each. Different formulae may be dispensed by mixing IRRP of different medicines and/or galenicals and/or herbal supplements and/or excipients, if needed, to obtain desired medical or nutritional preparations of desired weight or volume for taking by individual users. With the IRRP of different medical or nutritional preparations, a desired final medicine can be prepared in a more convenient manner and fine powder or dust produced during dispensing the formula can be minimized. The IRRP of medical or nutritional preparations also enable medicines and/or galenicals and/or herbal supplements to have increased stability and shelf life.

What is claimed is:

1. A method of dispensing a formula with medical or nutritional preparations in the form of initially-reduced-release particles (IRRP), comprising steps of using said at least one type of IRRP of medicines, galenicals or herbal supplements to dispense a formula and make up a final medical or nutritional preparation meeting a patient's or a user's individual need.

2. A method of dispensing a formula with medical or nutritional preparations in the form of initially-reduced-release particles (IRRP) as claimed in claim 1, wherein said at least one type of IRRP of medicines, galenicals or herbal supplements contains particles that are completely or mostly covered with one or more layers of coatings, thin films or other equivalent surroundings.

3. A method of dispensing a formula with medical or nutritional preparations in the form of initially-reduced-release particles (IRRP) as claimed in claim 1, wherein said at least one type of IRRP of medicines, galenicals or herbal supplements contains particles that have diameters within the range from about 0.05 mm to about 4 mm.

4. A method of dispensing a formula with medical or nutritional preparations in the form of initially-reduced-release particles (IRRP) as claimed in claim 2, wherein said coatings, thin films or other equivalent surroundings for said particles contained in said at least one type of IRRP of medicines, galenicals or herbal supplements are made of, but not limited to, non-water-soluble, low-water-soluble, acid-soluble, alkali-soluble, hydrogel forming, slowly-dissolving, biodegradable, hydrophilic, lipophilic, water-soluble, fat-soluble, and/or flavor-improving substance, and/or any other equivalent substance.

5. A method of dispensing a formula with medical or nutritional preparations in the form of initially-reduced-release particles (IRRP) as claimed in claim 2, wherein said coatings, thin films or other equivalent surroundings for said particles contained in said at least one type of IRRP of medicine, galenicals or herbal supplements are including, but not limited to, semi-permeable, permeable, gastric, enteric, erodible, slowly dissolving, biodegradable, low-water-soluble and/or partially dissolving in nature and/or have any other similar nature.

6. A method of dispensing a formula with medical or nutritional preparations in the form of initially-reduced-release particles (IRRP) as claimed in claim 1, wherein said at least one type of IRRP of medicines, galenicals or herbal supplements contains one or more types of medicines, galenicals or herbal supplements, and an amount of said one or more types of medicines, galenicals or herbal supplements that are released from said at least one type of IRRP in water of pH 7.0±0.1 at 37° C. within the first one minute does not exceed 10% of a total content of said medicines, galenicals or herbal supplements.

7. A method of dispensing a formula with medical or nutritional preparations in the form of initially-reduced-release particles (IRRP) as claimed in claim 1, wherein said at least one type of IRRP of medicines or galenicals or herbal supplements is mixed with predetermined amount of other type or types of initially-reduced-release or non-initially-reduced-release powders, granules, pellets, particles, etc. of drugs, galenicals nutriments, flavorings, sweetenings, herbal supplements, excipients, flavors, dietary supplements, foods, food additives, drug additives or other type or types of ingredients allowed to be used in preparing foods or drugs before, during or after making up said final medical or nutritional preparation.

8. A method of dispensing a formula with medical or nutritional preparations in the form of initially-reduced-release particles (IRRP) as claimed in claim 1, wherein said final medical preparation is divided by weight, by volume, by counting number of granules or by other predetermined dispensing means into doses matching with a a patient's or a user's individual needs.
